(19) United States
(12) Patent Application Publication
(10) Pub. No.: US 2023/0118672 A1
(43) Pub. Date: Apr. 20, 2023

(54) SUBSTITUTED POLY(ALKYLENE OXIDE) AND SURFACTANT COMPOSITION

(71) Applicant: SABIC Global Technologies B.V., Bergen op Zoom (NL)

(72) Inventors: Feng Xu, Riyadh (SA); Flaiyh Farhan N. Al-Anazi, Riyadh (SA)

(21) Appl. No.: 16/625,367

(22) PCT Filed: Jun. 19, 2018
(86) PCT No.: PCT/IB2018/054506

§ 371 Date: Dec. 20, 2019

Related U.S. Application Data
(60) Provisional application No. 62/523,052, filed on Jun. 21, 2017.

(51) Int. Cl. C08G 65/26; C11D 1/72; A61K 8/39

(52) U.S. Cl. CPC C08G 65/2609; C08G 65/2648; C08G 65/2696; C11D 1/72; A61K 8/39

(57) ABSTRACT

A method for the synthesis of a substituted poly(alkylene oxide) comprises reacting a substituted alcohol of formula (1) with an alkylene oxide of formula (2) in the presence of a catalyst and under conditions effective to provide the substituted poly(alkylene oxide) of formula (3) wherein in the foregoing formulas, each R is independently hydrogen, C₁₋₆₀ alkyl, or C₃₋₁₂ cycloalkyl, ring A is cyclohexane or phenyl, each R¹ is independently hydrogen, methyl, ethyl, propyl, butyl, hexyl, decyl, dodecyl, tetradecyl, or hexadecyl, preferably hydrogen or methyl, and n is 2 to 60.

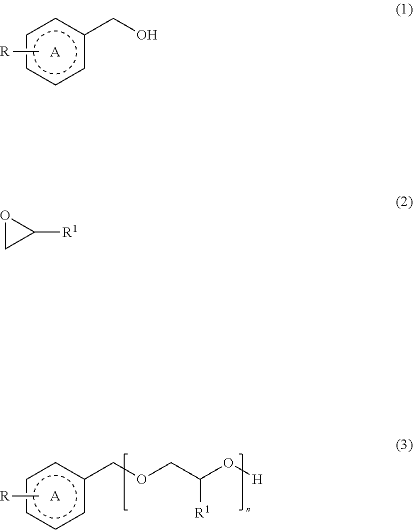

### BACKGROUND

This disclosure relates to a poly(alkylene oxide) surfactant, and in particular to a method for the synthesis of a substituted poly(alkylene oxide) surfactant.

Nonylphenol ethoxylate (NPE) is a non-ionic surfactant having excellent surfactant properties, low odor, low pour, and low chill points, and can be prepared and used at a lower cost compared to other non-ionic surfactants. However, NPE has been criticized for poor biodegradability, high aquatic toxicity resulting from biodegradation of phenol, and concern that NPE acts as an endocrine disrupter in humans. As a result, alkoxylated alkylphenols such as NPE have been banned in the European Union and voluntary restricted to industrial use in the United States.

There accordingly remains a need in the art for a non-ionic surfactant that can be a suitable substitute for NPE. It would be a further advantage if the non-ionic surfactant could be prepared by a convenient synthetic route.

### SUMMARY

According to an embodiment, a method for the synthesis of a substituted poly(alkylene oxide) comprises reacting a substituted alcohol of formula (1)

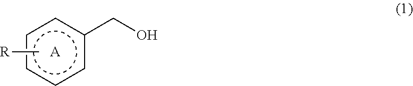

with an alkylene oxide of formula (2)

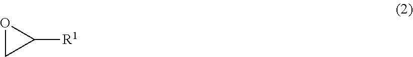

in the presence of a catalyst and under conditions effective to provide the substituted poly(alkylene oxide) of formula (3)

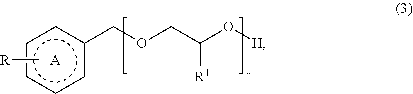

wherein in the foregoing formulas, each R is independently hydrogen, C₁₋₆₀ alkyl, or C₃₋₁₂ cycloalkyl, ring A is cyclohexane or phenyl, each R¹ is independently hydrogen, methyl, ethyl, propyl, butyl, hexyl, decyl, dodecyl, tetradecyl, or hexadecyl, preferably hydrogen or methyl, and n is 2 to 60.

According to another embodiment, a substituted poly(alkylene oxide) made by the method is provided.

In still another embodiment, a surfactant composition comprises the substituted poly(alkylene oxide).

In a further embodiment, a method for formulating and producing a cleaning product or a personal care product comprises using the substituted poly(alkylene oxide) or the surfactant composition.

The above described and other features are exemplified by the following drawings, detailed description, examples, and claims.

### DETAILED DESCRIPTION

This disclosure relates to a method for the synthesis of a substituted poly(alkylene oxide). The method provides a convenient and cost-effective method that is suitable for volume production of the substituted poly(alkylene oxide). The substituted poly(alkylene oxide) is particularly useful as a non-ionic surfactant as a substitute for nonylphenol ethoxylate (NPE) compounds. The substituted poly(alkylene oxide) demonstrates desirable surfactant properties including hydrophile-lipophile balance (HLB) number, critical micelle concentration (CMC), dynamic surface tension, and pour point that are comparable to NPE. Moreover, the substituted poly(alkylene oxide) is not an alkoxylated alkylphenol, and offers improved safety and environmental applicability over NPE. The substituted poly(alkylene oxide) can also be included in a surfactant composition. Both the substituted poly(alkylene oxide) and the surfactant composition can be useful in personal care products, industrial cleaners, electronics cleaners, automotive cleaners, home cleaners, food service cleaners, laundry detergents, dishwashing detergents, or other applications.

The poly(alkylene oxide) of the formula (A) is available via the synthetic route as shown in Scheme 1. However, the synthetic route is not suitable for volume production, therefore the poly(alkylene oxide)s have not been readily available for practical use.

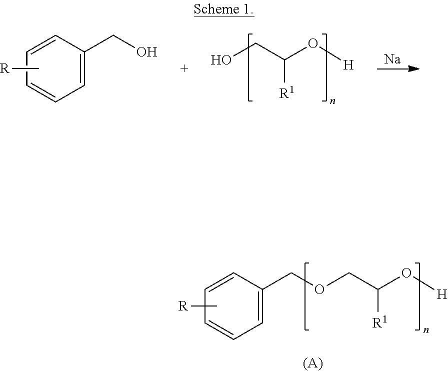

The inventors herein provide a synthetic method for the preparation of substituted poly(alkylene oxide)s, for example with an alkyl group substituted benzyl alcohol or cyclohexanemethanol as a chain starter. As an example, substituted poly(alkylene oxide)s can be prepared by reacting an alkylene oxide reagent with an alkyl group substituted benzyl alcohol or cyclohexanemethanol reagent in the presence of a catalyst, such as a base, double metal cyanide (DMC) catalyst, or calcium catalyst. The reaction is illustrated in Scheme 2.

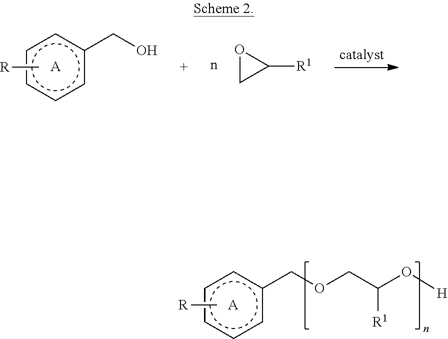

The substituted poly(alkylene oxide)s with structure of (A) or (B) can be prepared by an alkyl group substituted benzyl alcohol (C) or an alkyl group substituted cyclohexanemethanol (D), an alkylene oxide, and a catalyst, as shown in Scheme 3. The catalyst can include a base, a double metal cyanide (DMC), or a calcium compound.

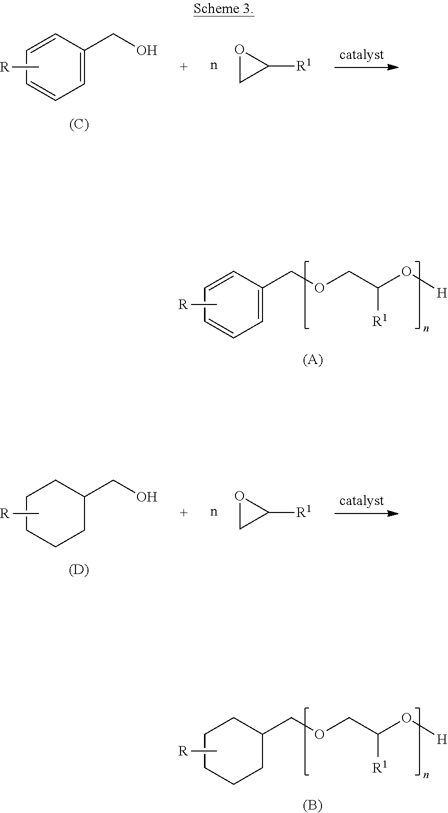

According to an exemplary embodiment, a method for the synthesis of a substituted poly(alkylene oxide) includes reacting a substituted alcohol of the formula (1)

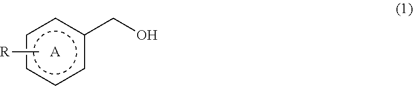

with an alkylene oxide of the formula (2)

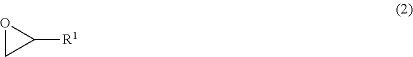

in the presence of a catalyst and under conditions effective to provide the substituted poly(alkylene oxide) of the formula (3)

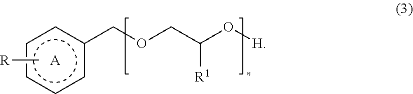

In formula (1), ring A is cyclohexane or phenyl. Each R is the same or different, and is independently hydrogen, C₁₋₆₀ alkyl, or C₃₋₁₂ cycloalkyl. In an embodiment, in formula (1), each R is independently hydrogen or C₁₋₁₆ alkyl, preferably C₁₀₋₁₆ alkyl, more preferably a C₁₂ alkyl or a C₁₆ alkyl. In another embodiment, in formula (1), each R is independently hydrogen or C₃₋₈ cycloalkyl, preferably cyclopentyl, cyclohexyl, or cycloheptyl. In some embodiments, ring A is cyclohexane or phenyl, and each R is independently hydrogen, C₁₋₁₆ alkyl, or C₃₋₈ cycloalkyl. In other embodiments, ring A is phenyl, and each R is independently hydrogen, C₁₀₋₁₆ alkyl, or C₅₋₇ cycloalkyl. In another embodiment, ring A is cyclohexane, and each R is independently hydrogen, C₁₀₋₁₆ alkyl, or C₅₋₇ cycloalkyl.

In the alkylene oxide of formula (2), each R¹ is independently hydrogen, methyl, ethyl, propyl, butyl, hexyl, decyl, dodecyl, tetradecyl, or hexadecyl, preferably hydrogen or methyl. In an embodiment, the alkylene oxide of formula (2) can be ethylene oxide, propylene oxide, ethyloxirane, propyloxirane, butyloxirane, hexyloxirane, decenoxirane, dodecyloxirane, tetradecyloxirane, hexadecyloxirane, or a combination comprising at least one of the foregoing.

In formula (3), ring A is cyclohexane or phenyl, each R¹ is independently hydrogen, methyl, ethyl, propyl, butyl, hexyl, decyl, dodecyl, tetradecyl, or hexadecyl, preferably hydrogen or methyl, each R is the same as in formula (1), and n is 2 to 60. In an embodiment, ring A is phenyl, each R¹ is independently hydrogen or methyl, each R is independently hydrogen or methyl, and n is 2 to 32. In another embodiment, ring A is cyclohexane, each R¹ is independently hydrogen or methyl, each R is independently hydrogen or methyl, and n is 2 to 32.

In an embodiment, the substituted poly(alkylene oxide) is of the formula (3a)

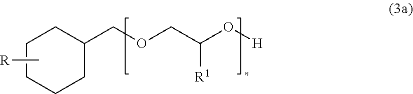

wherein in formula (3a), each R is independently hydrogen or C₁₋₁₆ alkyl, preferably C₁₀₋₁₆ alkyl, more preferably C₁₂ alkyl or C₁₆ alkyl. In formula (3a), each R¹ is independently hydrogen or methyl, and n is 2 to 32.

In another embodiment, the substituted poly(alkylene oxide) is one of the formulas

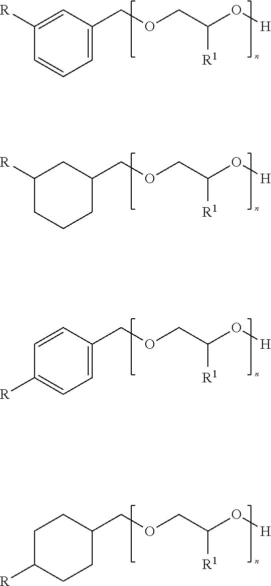

wherein in the formulas, each R is independently hydrogen or C₁₋₁₆ alkyl, preferably C₁₀₋₁₆ alkyl, more preferably C₁₂ alkyl or C₁₆ alkyl, each R¹ is independently hydrogen or methyl, and n is 2 to 60. In a particular embodiment, in the formulas, each R is independently C₁₂ alkyl or C₁₆ alkyl, each R¹ is independently hydrogen or methyl, and n is 2 to 48, preferably 2 to 40, more preferably 2 to 32.

In still another embodiment, the substituted poly(alkylene oxide) of one of the formulas

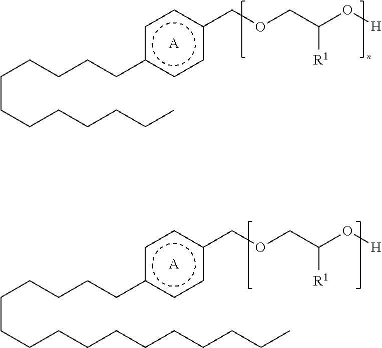

wherein in the formulas, ring A is cyclohexane or phenyl, each R¹ is independently hydrogen, methyl, ethyl, or propyl, preferably hydrogen or methyl. In an embodiment, ring A is phenyl, and each R¹ is independently hydrogen or methyl. In another embodiment, ring A is cyclohexane, and each R¹ is independently hydrogen or methyl.

The catalyst can include a base, a double metal cyanide, or a calcium compound. In an embodiment, the catalyst is a base that is an alkali or alkaline earth metal hydroxide, carbonate salt, carboxylic acid salt, oxide, or alkoxide salt. In an embodiment, the catalyst is sodium hydroxide, sodium methoxide, sodium ethoxide, magnesium oxide, potassium hydroxide, cesium hydroxide, strontium hydroxide, barium hydroxide, or barium oxide. In still another embodiment, the catalyst is sodium hydroxide or potassium hydroxide.

In an embodiment, the catalyst is a double metal cyanide compound of the formula (4)

M¹ₐ[M²(CN)_{b}L_{c}]_{d} (4)

wherein M¹ is Zn, Fe, Ni, Co, Mn, Sn, Pb, Mo, Al, V, Sr, W, Cu, or Cr, M² is Fe, Co, Cr, Mn, V, Ir, Ni, Rh, or Ru, and L is a halogen, NO, NO₂, CO, OH, H₂O, NCO, or NCS. In formula (4), a is 1 to 3, b is 5 or 6, c is 0 or 1, and d is 1 or 2. In an embodiment, M¹ is Zn and M² is Co. In still another embodiment, the catalyst is zinc hexacyanoferrate (III), zinc hexacyanoferrate (II), nickel (II) hexacyanoferrate (II), nickel (II) hexacyanoferrate (III), zinc hexacyanoferrate (III) hydrate, cobalt (II) hexacyanoferrate (II), nickel (II) hexacyanoferrate (III) hydrate, ferrous hexacyanoferrate (III), cobalt(II) hexacyanocobaltate (III), zinc hexacyanocobaltate (III), zinc hexacyanomanganate (II), zinc hexacyanochromate (III), zinc iodopentacyanoferrate (III), cobalt (II) chloropentacyanoferrate (II), cobalt (II) bromopentacyanoferrate (II), iron (II) fluoropentacyanoferrate (III), iron (III) hexacyanocobaltate (III), zinc chlorobromotetracyanoferrate (III), iron (III) hexacyanoferrate (III), aluminum dichlorotetracyanoferrate (III), molybdenum (IV) bromopentacyanoferrate (III), molybdenum (VI) chloropentacyanoferrate (II), vanadium (IV) hexacyanochromate (II), vanadium (V) hexacyanoferrate (III), strontium (II) hexacyanomanganate (III), tungsten (IV) hexacyanovanadate (IV), aluminum chloropentacyanovanadate (V), tungsten (VI) hexacyanoferrate (III), manganese (II) hexacyanoferrate (II), chromium (III) hexacyanoferrate (III), zinc hexacyanoiridate (III), nickel (II) hexacyaniridate (III), cobalt (II) hexacyanoiridate (III), ferrous hexacyanoiridate (III), or a combination comprising at least one of the foregoing. In a particular embodiment, the catalyst is zinc hexacyanocobaltate (III).

In an embodiment, the catalyst is a calcium compound, for example calcium oxide, calcium hydroxide, calcium sulfate, a calcium C₁₋₁₂ alkoxide, calcium acetate, calcium benzoate, calcium butyrate, calcium cinnamate, calcium citrate, calcium formate, calcium isobutyrate, calcium lactate, calcium laurate, calcium linoleate, calcium oleate, calcium palmitate, calcium propionate, calcium stearate, calcium valerate, calcium hexanoate, calcium octanoate, or a combination including at least one of the foregoing. In another embodiment, the catalyst is calcium hydroxide or calcium sulfate.

The catalyst can further include an activator. In an embodiment, the activator is an alcohol, an aldehyde, a ketone, an ether, an amide, a urea, a nitrile, a sulfide, or a combination including at least one of the foregoing. In an embodiment, the catalyst is a double metal cyanide compound of formula (4) and the activator is an alcohol. In a particular embodiment, the alcohol is ethanol, isopropyl alcohol, n-butyl alcohol, sec-butyl alcohol, tert-butyl alcohol, isobutyl alcohol, or a combination comprising at least one of the foregoing. In another embodiment, the catalyst is a double metal cyanide compound of formula (4) and the activator is tert-butyl alcohol. In still another embodiment, the activator is a C₁₋₆ alkyl ether of propylene glycol, dipropylene glycol, tripropylene glycol, ethylene glycol, diethylene glycol, triethylene glycol, or a combination comprising at least one of the foregoing. In yet another embodiment, the activator is a trihydroxy compound such as trimethylol propane or glycerin.

In an embodiment, conditions effective to provide the substituted poly(alkylene oxide) of formula (3) can include reaction parameters such as time, temperature, pressure, and catalyst amount. The reaction proceeds at acceptable rate, for example 1 to 360 minutes, if the temperature is 0 to 200° C., more specifically 20 to 180° C., even more specifically 40 to 150° C. The reaction is performed at a pressure which is atmospheric pressure or higher, for example less than 20 bar. Preferably, the pressure is 1 to 5 bar. In an embodiment, the catalyst is included in the reaction in an amount of 0.5 to 10 wt %, preferably 0.5 to 5 wt %, more preferably 0.5 to 3 wt %, based on the amount of alkylene oxide. The reaction can proceed with or without a solvent. When used, the solvent has no particular limit as long as it can dissolve or disperse the aforementioned components, but can include, for example, at least one of a ketone solvent such as methyl isobutyl ketone, 1-methyl-2-pyrrolidinone (NMP), cyclohexanone, acetone, or the like; an ether solvent such as tetrahydrofuran (THF); an acetate solvent such as ethyl acetate, butyl acetate, propylene glycol methyl ether acetate, or the like; an alcohol solvent such as isopropyl alcohol, butanol, or the like; or an amide solvent such as dimethylacetamide, dimethylformamide (DMF), or the like.

According to another embodiment, a method for the synthesis of the substituted alcohol of formula (1) is provided in Scheme 4:

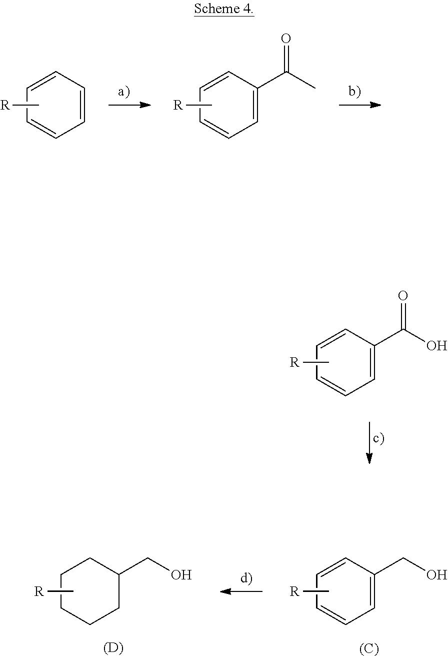

In Scheme 4, the reaction conditions are as follows: a) acetic anhydride or acetyl chloride, metal halide catalyst; b) oxygen and catalyst; c) hydrogen and catalyst; d) hydrogen and catalyst. More particular reaction conditions for these steps are provided below.

The method includes reacting a phenyl compound of the formula (5)

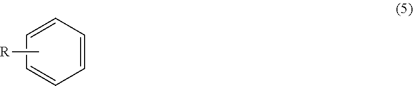

with acetic anhydride or acetyl chloride to provide an acetophenone compound of the formula (6)

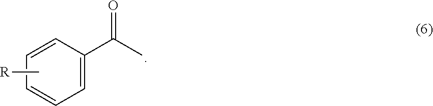

The Friedel-Crafts acylation reaction can be catalyzed by a metal catalyst, for example AlCl₃, AlBr₃, FeCl₃, or the like.

The acetophenone compound of the formula (6) can be oxidized to provide a substituted benzoic acid compound of the formula (7):

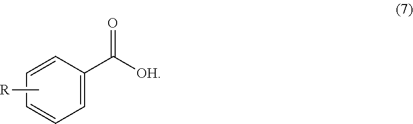

The oxidation reduction can be performed in the presence of ambient oxygen and catalyst. Suitable oxidation catalysts include V₂O₅, CO₃O₄, KMnO₄, K₂CrO₄, NaOCl, NaOBr, KOCl, KOBr, KOI, or the like.

The substituted benzoic acid compound of the formula (7) can then be reduced to provide a substituted alcohol of formulas (1a) or (1b):

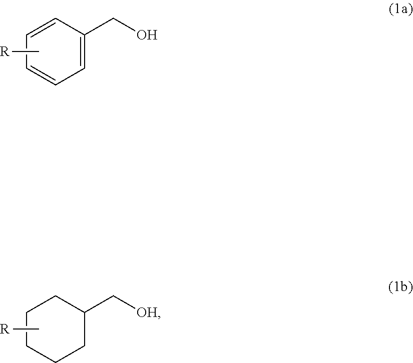

or a combination comprising at least one of the foregoing. The formation of the substituted alcohol of formula (1a) can include, for example, reduction using hydrogen and a catalyst, for example palladium on carbon (Pd/C), lithium aluminum hydride, or the like. The substituted alcohol of formula (1a) can be further reduced to provide the substituted alcohol of formula (1b), for example, using hydrogen and a catalyst, for example PtO₂, Pt/C, or the like. The substituted alcohols of the formulas (1a) and (1b) are collectively referred to herein as “chain starters” because they are a point from which the poly(alkylene oxide) extends.

In another exemplary embodiment, a substituted poly(alkylene oxide) made by the methods disclosed herein is provided. In an embodiment, the substituted poly(alkylene oxide) has at least one of a hydrophile-lipophile balance (HLB) number of 7 to 14, preferably 8 to 12, more preferably 9 to 11; a critical micelle concentration (CMC) of 0.01 to 5%, preferably 0.01 to 3%, more preferably 0.01 to 1%, as measured at 25° C.; a dynamic surface tension of 10 to 75 dynes per centimeter (dyne/cm), preferably 10 to 50 dyne/cm, more preferably 10 to 40 dyne/cm, as measured at 25° C.; a pour point of −40 to 20° C., preferably −30 to 10° C., more preferably −20 to 0° C.; or a viscosity of 20 to 50 centipoise (cP), preferably 20 to 40 cP, more preferably 25 to 35 cP, as measured at 25° C.

According to another exemplary embodiment, a surfactant composition includes the substituted poly(alkylene oxide) made by the methods disclosed herein. In an embodiment, the surfactant composition comprises the substituted poly(alkylene oxide) described herein in an amount of 0.1 to 50 weight percent, based on a total weight of the surfactant composition. In an embodiment, the surfactant composition is a laundry detergent, dish detergent, dishwasher detergent, industrial cleaning fluid, home cleaning fluid, biodegradable cleaning fluid, fabric cleaning fluid, floor cleaning fluid, hand cleaning fluid, body wash, medical cleaning fluid, kitchen cleaning fluid, oven cleaning fluid, or surface cleaning fluid.

The surfactant composition can further include an auxiliary surfactant, a solvent, an adjunct polymer or copolymer, an enzyme, an enzyme stabilizer, a viscosity regulator, a bleach, a hydrotope, a suds boosters, a suds suppressors (antifoams), dispersant, silvercare, anti-tarnish and/or anti-corrosion agents, an inorganic salt, a fragrance, a dye, a pigment, a color speckle, a filler, a germicide, an alkalinity source, an antioxidant, a carrier, a processing aid, a buffer, a chelating agent, a dye transfer inhibiting agent, a fabric softener, an anti-abrasion agent, a preservative, a nutrient, a moisturizer, an emollient, an aqueous phase component, or a combination comprising at least one of the foregoing.

The components of the surfactant composition can be incorporated at levels sufficient to provide a “cleaning-effective amount”. The term “cleaning effective amount” refers to any amount capable of producing a cleaning, stain removal, soil removal, degreasing, whitening, deodorizing, disinfecting, or freshness improving effect on substrates such as fabrics, nonporous surfaces, metal parts, dishware, skin, or the like.

Suitable solvents include mono- and/or polyfunctional alcohols having from 1 to 6 carbon atoms. Preferred alcohols are ethanol, 1,2-propanediol, glycerol, or a combination comprising at least one of the foregoing. The compositions contain preferably from 2 to 20 weight percent (wt %) of the solvent, and in particular from 5 to 15 wt % of ethanol or any mixture of ethanol and 1,2-propanediol or, in particular, of ethanol and glycerol, based on the total weight of the composition. The compositions can further include polyethylene glycol having a relative molecular mass of between 200 and 2,000 grams per mole (g/mol), preferably up to 600 g/mol, in amounts of from 2 to 17 wt %, based on the total weight of the surfactant composition.

In an embodiment, the surfactant composition can include an adjunct polymer or copolymer. Suitable examples include, but are not limited to, polyvinyl pyrrolidone (PVP); polyethyleneglycol dimethylether (DM-PEG); vinylpyrrolidone/dialkylaminoalkyl acrylate or methacrylate; a polystyrenesulphonate (PSS); polyvinyl pyridine-N-oxide (PVNO); polyvinylpyrrolidone/vinylimidazole (PVP-VI); polyvinylpyrrolidone/polyacrylic acid (PVP-AA); polyvinylpyrrolidone/vinylacetate (PVP-VA); polyacrylate or polyacrylicmalate; or a combination comprising at least one of the foregoing. The adjunct polymer or copolymer can be included in an amount of 0.005 to 5.0 wt %, preferably 0.10 to 4.0 wt %, more preferably from 0.1% to 3.0 wt %, based on the total weight of the surfactant composition.

Suitable hydrotropes that can be used include toluenesulfonate, xylenesulfonate, cumenesulfonate, or a combination comprising at least one of the foregoing.

In an embodiment, antioxidants such as carbamate, ascorbate, organic amines such as ethylenediaminetetracetic acid (EDTA) or alkali metal salt thereof, monoethanolamine (MEA), or a combination comprising at least one of the foregoing can be used. The antioxidants can further include chlorine scavenger anions including salts containing ammonium cations with sulfite, bisulfite, thiosulfite, thiosulfate, iodide, or the like.

Bleaches can yield hydrogen peroxide in water, and include, for example, sodium perborate tetrahydrate, sodium perborate monohydrate, peroxycarbonate, citrate perhydrates, and salts of peracids, such as perbenzoates, peroxyphthalates, or diperoxydodecanedioic acid. Bleaches and bleaching agents other than oxygen bleaching agents are also known in the art and can be utilized herein. In an embodiment, a photoactivated bleaching agent such as sulfonated zinc and/or aluminum phthalocyanine can be used. In another embodiment, the bleaching compounds can be catalyzed by means of a manganese compound or a cobalt compound. The bleaches can be included in amounts of from 0.025 to 25 wt %, based on the total weight of the surfactant composition.

Viscosity regulators include hydrogenated castor oil, salts of long-chain fatty acids, which are used preferably in amounts of from 0 to 5 wt %, based on the total weight of the surfactant composition, examples being sodium, potassium, aluminum, magnesium, and titanium stearates, or the sodium or potassium salts of behenic acid, and also further polymeric compounds, for example polyvinylpyrrolidone, urethanes, and the salts of polymeric polycarboxylates, examples including homopolymeric or copolymeric polyacrylates, polymethacrylates and, in particular, copolymers of acrylic acid with maleic acid. The molecular mass of the homopolymers can be 1,000 and 100,000 g/mol, the copolymers can be 2,000 and 200,000 g/mol, based on the free acid. Also suitable are water-soluble polyacrylates that are crosslinked, for example, with 1 wt % of a polyallyl ether of sucrose. Examples include the polymers having a thickening action which are obtainable under the name CARBOPOL® 940 and 941. The compositions can further comprise from 5 to 20 wt % of partially esterified copolymers, for example as obtained by copolymerizing (a) at least one C₄-C₂₈ olefin or mixtures of at least one C₄₋₂₈ olefin with up to 20 mol % of C₁₋₂₈ alkyl vinyl ethers and (b) ethylenically unsaturated dicarboxylic anhydrides having from 4 to 8 carbon atoms in a molar ratio of 1:1, and then partially esterifying the copolymers with reaction products such as C₁₋₁₃ alcohols, C₈₋₂₂ fatty acids, C₁₋₁₂ alkylphenols, secondary C₂₋₃₀ amines, or a combination comprising at least one of the foregoing, with at least one C₂₋₄ alkylene oxide or tetrahydrofuran, and hydrolyzing the anhydride groups of the copolymers to carboxyl groups, the partial esterification of the copolymers being conducted to an extent such that from 5 to 50% of the carboxyl groups of the copolymers are esterified. The partially esterified copolymers then can be present either in the form of the free acid or, preferably, in partly or fully neutralized form. The compositions can include partially esterified copolymers in amounts of from 5 to 15 wt %, and in particular 8 to 12 wt %, based on the total weight of the surfactant composition.

Suitable fragrances include extracts and essences which can comprise complex mixtures of natural ingredients, such as orange oil, lemon oil, rose extract, lavender, musk, patchouli, balsamic essence, sandalwood oil, pine oil, cedar, Peru balsam, Olibanum resinoid, styrax, labdanum resin, nutmeg, cassia oil, benzoin resin, coriander, lavandinor, or the like. Non-limiting examples of fragrance ingredients include: 7-acetyl-1,2,3,4,5,6,7,8-octahydro-1,1,6,7-tetramethyl naphthalene; ionone methyl; ionone gamma methyl; methyl cedrylone; methyl dihydrojasmonate; methyl 1,6,10-trimethyl-2,5,9-cyclododecatrien-1-yl ketone; 7-acetyl-1,1,3,4,4,6-hexamethyl tetralin; 4-acetyl-6-tert-butyl-1,1-dimethyl indane; para-hydroxy-phenyl-butanone; benzophenone; methyl beta-naphthyl ketone; 6-acetyl-1,1,2,3,3,5-hexamethyl indane; 5-acetyl-3-isopropyl-1,1,2,6-tetramethyl indane; 1-dodecanal, 4-(4-hydroxy-4-methylpentyl)-3-cyclohexene-1-carboxaldehyde; 7-hydroxy-3,7-dimethyl ocatanal; 10-undecen-1-al; iso-hexenyl cyclohexyl carboxaldehyde; formyl tricyclodecane; condensation products of hydroxycitronellal and methyl anthranilate, condensation products of hydroxycitronellal and indol, condensation products of phenyl acetaldehyde and indol; 2-methyl-3-(para-tert-butylphenyl)-propionaldehyde; ethyl vanillin; heliotropin; hexyl cinnamic aldehyde; amyl cinnamic aldehyde; 2-methyl-2-(para-iso-propylphenyl)-propionaldehyde; coumarin; decalactone gamma; cyclopentadecanolide; 16-hydroxy-9-hexadecenoic acid lactone; 1,3,4,6,7,8-hexahydro4,6,6,7,8,8-hexamethylcyclopenta-gamma-2-benzo-pyrane; beta-naphthol methyl ether; ambroxane; dodecahydro-3a,6, 6,9a-tetramethyl-naphtho[2,1]furan; cedrol, 5-(2,2,3-trimethylcyclopent-3-enyl)-3-methylpentan-2-ol; 2-ethyl-4-(2,2,3-trimethyl-3-cyclopenten-1-yl)-2-buten-1-ol; caryophyllene alcohol; tricyclodecenyl propionate; tricyclodecenyl acetate; benzyl salicylate; cedryl acetate; and para-(tert-butyl) cyclohexyl acetate; hexyl cinnamic aldehyde; 2-methyl-3-(para-tert-butylphenyl)-propionaldehyde; 7-acetyl-1,2,3,4,5,6,7,8-octahydro-1,1,6,7-tetramethyl naphthalene; benzyl salicylate; 7-acetyl-1,1,3,4,4,6-hexamethyl tetralin; para-tert-butyl cyclohexyl acetate; methyl dihydro jasmonate; beta-napthol methyl ether; methyl beta-naphthyl ketone; 2-methyl-2-(para-iso-propylphenyl)-propionaldehyde; 1,3,4,6,7,8-hexahydro-4,6,6,7,8, 8-hexamethyl-cyclopenta-gamma-2-benzopyrane; dodecahydro-3a,6,6,9a-tetramethylnaphtho[2,1b]furan; anisalde-hyde; coumarin; cedrol; vanillin; cyclopentadecanolide; tricyclodecenyl acetate; and tricyclodecenyl propionate.

In an embodiment, suitable enzymes include proteases, lipases, amylases, cellulases, cutinases, or a combination comprising at least one of the foregoing. The compositions can comprise further enzyme stabilizers. For example, from 0.5 to 1 wt % of sodium formate can be used. In an embodiment, proteases stabilized with soluble calcium salts, with a calcium content of preferably 1.2% by weight, based on the weight of the enzyme, can be used. Enzyme stabilizers include boric acid, boron oxide, borax, and other alkali metal borates such as the salts of orthoboric acid (H₃BO₃), of metaboric acid (HBO₂), and of pyroboric acid (tetraboric acid, H₂B₄O₇).

In an embodiment, the surfactant composition can further include a chelating agent. Suitable chelating agents include amino carboxylates, amino phosphonates, polyfunctionally-substituted aromatic chelating agents, or a combination comprising at least one of the foregoing. For example, the chelating agent is ethylene diaamine tetracetate, N-hydroxyethylethylene diamine triacetate, nitrilotriacetate, ethylene diamine tetrapropionate, triethylene tetraamine hexacetate, diethylene triamine pentaacetate, ethanol diglycine, or alkali metal, ammonium, and substituted ammonium salts thereof; dihydroxydisulfobenzenes such as 1,2-dihydroxy-3,5-disulfobenzene; a biodegradable chelator such as ethylenediamine disuccinate or methyl glycine diacetic acid salts. A combination comprising at least one of the foregoing chelating agents can be used.

Suitable foam inhibitors include soaps of natural or synthetic origin containing a high proportion of C₁₈₋₂₄ fatty acids, organopolysiloxanes and their mixtures with microfine silica, paraffins, waxes, microcrystalline waxes, and their mixtures with silanized silica or bistearylethylenediamide, or a combination comprising at least one of the foregoing.

In an embodiment, the surfactant compositions can further include one or more auxiliary surfactants. Suitable auxiliary surfactants include, but are not limited to, anionic surfactants, preferably alkyl alkoxylated sulfates, alkyl sulfates, and/or linear alkyl benzenesulfonate surfactants; cationic surfactants, preferably quaternary ammonium surfactants; nonionic surfactants, preferably alkyl ethoxylates, alkyl polyglucosides, and/or amine or amine oxide surfactants; amphoteric surfactants, preferably betaines and/or polycarboxylates (for example polyglycinates); and zwitterionic surfactants.

Suitable aqueous phase components include, but are not limited to, amino acids such as glycine, alanine, serine, threonine, arginine, glutamic acid, aspartic acid, leucine, valine, or the like; polyalcohols such as glycerin, ethylene glycol, 1,3-butylene glycol, propylene glycol, isoprene glycol, or the like; water-soluble polymers such as polyamino acids including polyglutamic acid and polyaspartic acid and their salts, polyethylene glycol, arabic gum, alginic acid salts, xanthan gum, hyaluronic acid, salts of hyaluronic acid, chitin, chitosan, water-soluble chitin, carboxyvinyl polymers, carboxymethyl cellulose, hydroxyethyl cellulose, hydroxypropyltrimethylammonium chloride, polydimethylmethylenepiperidium chloride, quaternary ammonium salts of polyvinylpyrrolidone derivatives, cationated proteins, collagen decomposates and their derivatives, acylated proteins, polyglycerins, amino acid polyglycerin esters, or the like; glycoalcohols such as mannitol and their alkylene oxide adducts; as well as lower alcohols such as ethanol, propanol, or the like.

In another embodiment, the surfactant composition can further include any of a variety of other ingredients suitable to produce a surfactant composition with additional functional or cosmetic benefits. Such ingredients include but are not limited to: (a) non-ethoxylated non-ionic surfactants other than polyglyceryl non-ionic surfactants, including but not limited to, alkyl polyglucosides (e.g., decyl glucoside, coco-glucoside, lauryl glucoside), alkyl polypentosides (e.g., caprylyl/capryl wheat bran/straw glycosides), sucrose esters (e.g., sucrose cocoate, sucrose laurate), sorbitan esters (e.g., sorbitan laurate, sorbitan caprylate), or a combination comprising at least one of the foregoing, or the like; (b) rheology modifiers, including but not limited to, naturally-derived polysaccharides including xanthan gum, dehydroxanthan gum, guar gum, cassia gum, carrageenan gum, alginic acid, and alginate gums (e.g., algin, calcium alginate), gellan gum, pectin, microcrystalline cellulose, non-ethoxylated derivatives of cellulose (e.g., sodium carboxymethylcellulose, hydroxypropyl methylcellulose), and hydroxypropyl guar, and synthetic polymers that do not comprise ethoxylated monomers or ethoxylated surfactants (e.g., as processing or dispersing aids), for example, acrylic alkali-swellable emulsion (ASE) polymers, such as Acrylates Copolymer, available under the trade name CARBOPOL AQUA SF-1 from Lubrizol Corp., hydrophobically-modified acrylate crosspolymers, such as Acrylates/C10-30 Alkyl Acrylates Crosspolymer, available under the trade name CARBOPOL 1382 from Lubrizol Corp., non-ethoxylated micellar thickeners, such as: cocamide MIPA, lauryl lactyl lactate, or sorbitan sesquicaprylate, or a combination comprising at least one of the foregoing, or the like; (c) conditioning agents including but not limited to cationic surfactants, cationic polymers, such as, cationically-modified polysaccharides, including starch hydroxypropyltrimonium chloride, guar hydroxypropyltrimonium chloride, and hydroxypropyl guar hydroxypropyltrimonium chloride, cationic polymers derived from the (co)polymerization of ethylenically-unsaturated cationic monomers with optional hydrophilic monomers, including polyquaternium-5, polyquaternium-6, polyquaternium-7, polyquaternium-11, polyquaternium-14, polyquaternium-15, polyquaternium-28, polyquaternium-39, polyquaternium-44; polyquaternium-76; or a combination comprising at least one of the foregoing, or the like; and (d) other preservatives and preservative boosters including but not limited to benzyl alcohol, caprylyl glycol, decylene glycol, ethylhexylglycerin, gluconolactone, methylisothazolinone, or a combination comprising at least one of the foregoing, or the like.

In certain embodiments, the surfactant composition can be used as or in personal care products for treating or cleansing at least a portion of the human body. Examples of certain preferred personal care products include various products suitable for application to the skin, hair, or genital region of the body, such as shampoos, hand, face, and/or body washes, bath additives, gels, lotions, creams, and the like. In another embodiment, the surfactant composition can be used as or in cleaning products for cleaning non-porous surfaces. For example, the surfactant composition can be used in automotive detergents, textile scouring agents, metal surface-treatment agents, metal degreasing agents, detergents for metallic parts, detergents for electronic parts, leather detergents, depitching agents, detergents for linen supplies (laundry detergents), kitchen detergents, fingertip detergents, and dry-cleaning additives, or the like.

The hydrophile-lipophile balance “HLB” number of a compound or composition denotes the relative simultaneous attraction that is demonstrated for water and oil. Thus, substances having a high HLB value above 12 are highly hydrophilic (and poorly lipophilic), while substances having a low HLB value, below 8, are lipophilic and consequently poorly hydrophilic. Substances having an HLB value of between 8 and 12 are intermediate. The HLB number can be calculated based on non-ionic surfactant blend standards having an HLB of 2 to 16.

The critical micelle concentration (CMC) can be measured according to the ISO 4311:1979 standard.

Dynamic surface tension can be measured for aqueous solutions of various compounds using the maximum bubble pressure method at bubble rates from 0.1 bubbles per second (b/s) to 20 b/s. These data provide information about the performance of a surfactant at conditions from near-equilibrium (0.1 b/s) through extremely high surface creation rates (20 b/s).

Pour point can be measured according to the ASTM D97 standard test method.

Viscosity can be measured according to the ASTM D445 standard test method.

This disclosure is further illustrated by the following examples, which are non-limiting.

### EXAMPLES

All reagents were obtained from a commercial source (e.g., Sigma Aldrich) and used as received unless noted otherwise.

### Example 1

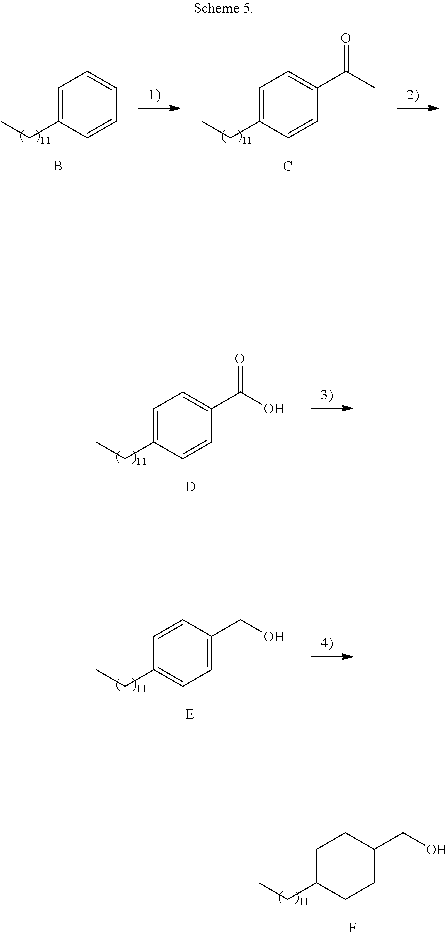

Scheme 5 illustrates an exemplary reaction sequence for the synthesis of 4-dodecylcyclohexylmethanol (F). Each reaction step is provided below.

### Step 1: Synthesis of 4-dodecylacetophenone (C)

Aluminum chloride (113.56 grams (g), 0.85 moles (mol)) and methylene chloride (400 mL) were added into a three-necked round bottom flask successively under nitrogen atmosphere. The suspension was cooled to 0° C. To this, a solution of acetic anhydride (44.94 g, 0.44 mol)-methylene chloride (100 mL) was slowly added dropwise within a minimum period of 30 minutes. The resulting mixture was stirred for about 15 minutes (min) and subsequently a cold (0° C.) dodecylbenzene (B) (49.63 g, 0.2 mol)-methylene chloride solution was added dropwise into the round bottom flask under cooling within a period of 30 min. The reaction mixture was then stirred overnight and the temperature gradually rose to room temperature (ca. 23° C.). The resulting reaction system was poured slowly onto 1.5 L of crushed ice, after which the ice melted. The aqueous phase was separated and discarded. The organic phase was washed with hydrochloric acid (2×500 mL, 3 molar (M)), a saturated sodium carbonate solution, and a saturated sodium chloride solution. The organic phase was then dried with anhydrous magnesium sulfate overnight, and the solvent was removed on a rotary evaporator. The resulting brown solid was purified by recrystallization from methanol overnight at 4° C. The obtained product, 4-dodecylacetophenone (C), was 53.33 g with a yield of 92%.

### Step 2: Synthesis of 4-dodecylbenzoic acid (D)

A sodium hypochlorite aqueous solution (10%, 1,200 g) and sodium hydroxide (80 g, 2 mol) were added into a 2,000 mL four-neck round bottom flask. The system was heated at 40-50° C. and then 4-dodecylacetophenone (C) (116.68 g, 0.4 mol) was added dropwise within a period of 2 hours. The reaction system was heated gradually up to 75° C. and then stirred at 75° C. for 2 hours. After that, the system was cooled to a temperature below 30° C. and an excess of a sodium sulfite aqueous solution was added into the round bottom flask. After stirring for 1 hour, concentrated hydrochloric acid was gradually added, resulting in precipitation of the 4-dodecylbenzoic acid (D). After cooling and filtration, the obtained solid was recrystallized from ethanol. The obtained product was 92.86 g with a yield of 80%.

### Step 3: Synthesis of 4-dodecylbenzyl Alcohol (E)

Lithium aluminum hydride (11.3 g, 0.298 mol) was suspended in anhydrous ethyl ether (350 mL) under nitrogen and chilled at 0° C. A solution of 4-dodecylbenzoic acid (D) (24.64 g, 0.085 mol) in anhydrous ethyl ether was added dropwise to the reaction system over 4 hours while the temperature was maintained at 0° C. The reaction was then allowed to warm to room temperature (ca. 23° C.), and stirring was continued overnight at room temperature. The reaction was then quenched with potassium hydrogen sulfate (57.2 g, 0.42 mol) in 600 mL of water. The reaction mixture was extracted with ethyl ether (3×300 mL). The combined organic extracts were washed with 3 normality (N) hydrochloric acid (3×250 mL), a saturated sodium bicarbonate aqueous solution, water, and finally with a saturated sodium chloride aqueous solution. The organic extract was dried over anhydrous magnesium sulfate, filtered, and concentrated to remove the organic solvent. The remainder was subjected to vacuum distillation and the fraction at 178-180° C./1,000 Pa was collected as the product 4-dodecylbenzyl alcohol (E). The obtained product was 17.6 g with a yield of 75%.

### Step 4: Synthesis of 4-dodecylcyclohexylmethanol (F)

A high-pressure autoclave was charged with 4-dodecylbenzyl alcohol (E) (13.8 g, 0.05 mol), water (2,500 mL), and a Pt catalyst (500 mg). Afterwards, the autoclave was successively pressurized with H₂ (4 bar) followed by evacuation three times to displace residual air in the reactor. The autoclave was then filled with H₂ (60 bar), heated at 100° C., and kept at that temperature for 6 hours. After that, the autoclave was cooled to room temperature (ca. 23° C.). The reaction mixture was filtered and extracted with ethyl ether (3×300 mL). The combined organic extracts were dried over anhydrous magnesium sulfate, filtered, and concentrated to remove the organic solvent. The remainder was the product 4-dodecylcyclohexylmethanol (F). The obtained product was 13.4 g with a yield of 95%.

### Example 2

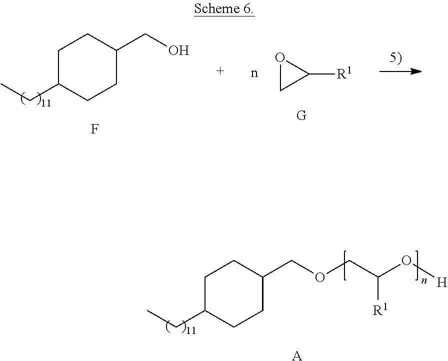

### Step 5: Synthesis of 4-dodecylcyclohexylmethanol-(EO)₁₀H (R¹═H)

The reaction sequence is illustrated in Scheme 6. As obtained from Example 1, 4-Dodecylcyclohexylmethanol (F) (7.1 g, 25 mmol) was mixed in an autoclave with KOH (40 mg) and reacted with ethylene oxide (G) (11 g, 250 mmol) fed into the autoclave at a temperature of 150° C. and at a pressure of 3.0 bar. The pressure and temperature were controlled by the ethylene oxide charge rate and rate of flow of the cooling water. After charging with ethylene oxide, the reaction was held for half an hour and the autoclave was then placed under a 50 mm vacuum for thirty minutes, and then the vacuum was broken with nitrogen. The resulting product, 4-dodecylcyclohexylmethanol-(EO)₁₀H (A), was cooled at 40° C. and the catalyst was neutralized with acetic acid to a pH of 6 to 7. The obtained product was 18.2 g.

This disclosure further encompasses the following aspects.

Aspect 1: A method for the synthesis of a substituted poly(alkylene oxide) comprises reacting a substituted alcohol of formula (1)

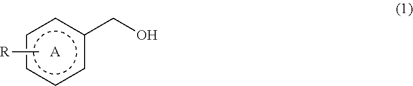

with an alkylene oxide of formula (2)

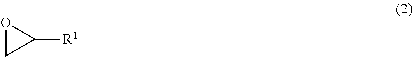

in the presence of a catalyst and under conditions effective to provide the substituted poly(alkylene oxide) of formula (3)

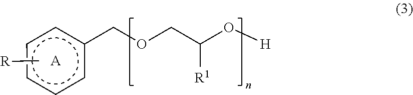

wherein in the foregoing formulas, each R is independently hydrogen, C₁₋₆₀ alkyl, or C₃₋₁₂ cycloalkyl, ring A is cyclohexane or phenyl, each R¹ is independently hydrogen, methyl, ethyl, propyl, butyl, hexyl, decyl, dodecyl, tetradecyl, or hexadecyl, preferably hydrogen or methyl, and n is 2 to 60.

Aspect 2: The method of Aspect 1, wherein the catalyst is a base, a double metal cyanide, or a calcium compound.

Aspect 3: The method of Aspect 1 or 2, wherein the catalyst is sodium hydroxide, sodium methoxide, sodium ethoxide, magnesium oxide, potassium hydroxide, cesium hydroxide, strontium hydroxide, barium hydroxide, or barium oxide; or the catalyst is a double metal cyanide compound of formula (4)

M¹ₐ[M²(CN)_{b}L_{c}]_{d} (4)

wherein M¹ is Zn, Fe, Ni, Co, Mn, Sn, Pb, Mo, Al, V, Sr, W, Cu, or Cr, preferably Zn, M² is Fe, Co, Cr, Mn, V, Ir, Ni, Rh, or Ru, preferably Co, L is a halogen, NO, NO₂, CO, OH, H₂O, NCO, or NCS, a is 1 to 3, b is 5 or 6, c is 0 or 1, and d is 1 or 2; or the catalyst is calcium oxide, calcium hydroxide, calcium sulfate, a calcium alkoxide, calcium acetate, calcium benzoate, calcium butyrate, calcium cinnamate, calcium citrate, calcium formate, calcium isobutyrate, calcium lactate, calcium laurate, calcium linoleate, calcium oleate, calcium palmitate, calcium propionate, calcium stearate, calcium valerate, calcium hexanoate, calcium octanoate, or a combination comprising at least one of the foregoing.

Aspect 4: The method of any one or more of Aspects 1 to 3, wherein the catalyst is zinc hexacyanoferrate (III), zinc hexacyanoferrate (II), nickel (II) hexacyanoferrate (II), nickel (II) hexacyanoferrate (III), zinc hexacyanoferrate (III) hydrate, cobalt (II) hexacyanoferrate (II), nickel (II) hexacyanoferrate (III) hydrate, ferrous hexacyanoferrate (III), cobalt(II) hexacyanocobaltate (III), zinc hexacyanocobaltate (III), zinc hexacyanomanganate (II), zinc hexacyanochromate (III), zinc iodopentacyanoferrate (III), cobalt (II) chloropentacyanoferrate (II), cobalt (II) bromopentacyanoferrate (II), iron (II) fluoropentacyanoferrate (III), iron (III) hexacyanocobaltate (III), zinc chlorobromotetracyanoferrate (III), iron (III) hexacyanoferrate (III), aluminum dichlorotetracyanoferrate (III), molybdenum (IV) bromopentacyanoferrate (III), molybdenum (VI) chloropentacyanoferrate (II), vanadium (IV) hexacyanochromate (II), vanadium (V) hexacyanoferrate (III), strontium (II) hexacyanomanganate (III), tungsten (IV) hexacyanovanadate (IV), aluminum chloropentacyanovanadate (V), tungsten (VI) hexacyanoferrate (III), manganese (II) hexacyanoferrate (II), chromium (III) hexacyanoferrate (III), zinc hexacyanoiridate (III), nickel (II) hexacyaniridate (III), cobalt (II) hexacyanoiridate (III), ferrous hexacyanoiridate (III), or a combination comprising at least one of the foregoing.

Aspect 5. The method of any one or more of Aspects 1 to 4, wherein the catalyst further comprises an activator that is an alcohol, an aldehyde, a ketone, an ether, an amide, a urea, a nitrile, a sulfide, or a combination comprising at least one of the foregoing.

Aspect 6: The method of any one or more of Aspects 1 to 5, wherein each R is independently hydrogen or C₁₋₁₆ alkyl, preferably C₁₀₋₁₆ alkyl, more preferably C₁₂ alkyl or C₁₆ alkyl.

Aspect 7: The method of any one or more of Aspects 1 to 5, wherein each R is independently hydrogen or C₃₋₈ cycloalkyl, preferably cyclopentyl, cyclohexyl, or cycloheptyl.

Aspect 8: The method of any one or more of Aspects 1 to 6, wherein the substituted poly(alkylene oxide) is of formula (3a)

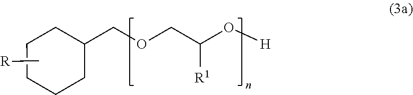

wherein each R is independently hydrogen or C₁₋₁₆ alkyl, preferably C₁₀₋₁₆ alkyl, more preferably C₁₂ alkyl or C₁₆ alkyl, each R¹ is independently hydrogen or methyl, and n is 2 to 32.

Aspect 9: The method of any one or more of Aspects 1 to 8, further comprising reacting a phenyl compound of formula (5)

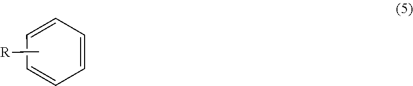

with acetic anhydride or acetyl chloride to provide an acetophenone compound of formula (6)

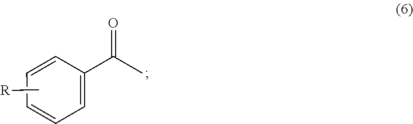

oxidizing the acetophenone compound to provide a substituted benzoic acid compound of formula (7)

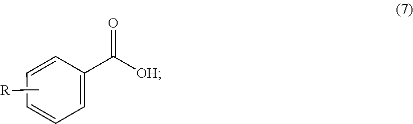

and
reducing the substituted benzoic acid compound to provide the substituted alcohols of formulas (1a) or (1b):

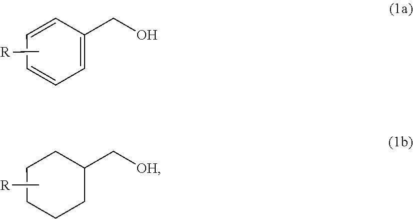

or
a combination comprising at least one of the foregoing.

Aspect 10: A substituted poly(alkylene oxide) made by any one or more of the methods of Aspects 1 to 9.

Aspect 11: The substituted poly(alkylene oxide) of Aspect 10, wherein the substituted polyalkylene has at least one of: a hydrophile-lipophile balance number of 7 to 14, preferably 8 to 12, more preferably 9 to 11; a critical micelle concentration of 0.01 to 5%, preferably 0.01 to 3%, more preferably 0.01 to 1%, as measured at 25° C.; a dynamic surface tension of 10 to 75 dynes per centimeter, preferably 10 to 50 dynes per centimeter, more preferably 10 to 40 dynes per centimeter, as measured at 25° C.; a pour point of −40 to 20° C., preferably −30 to 10° C., more preferably −20 to 0° C.; or a viscosity of 20 to 50 centipoise, preferably 20 to 40 centipoise, more preferably 25 to 35 centipoise, as measured at 25° C.

Aspect 12: A surfactant composition comprising the substituted poly(alkylene oxide) of Aspect 10 or 11, or made by any one or more of the methods of Aspects 1 to 9.

Aspect 13: The surfactant composition of Aspect 12, wherein the surfactant composition comprises the substituted poly(alkylene oxide) in an amount of 0.1 to 50 weight percent, based on a total weight of the surfactant composition.

Aspect 14: The surfactant composition of Aspect 12 or 13, further comprising an auxiliary surfactant, a solvent, an enzyme, an enzyme stabilizer, a viscosity regulator, a bleach, a hydrotope, an inorganic salt, a fragrance, a dye, a buffer, a preservative, a nutrient, a moisturizer, an emollient, or a combination comprising at least one of the foregoing.

Aspect 15: A method for formulating and producing a cleaning product or a personal care product, the method comprising using the substituted poly(alkylene oxide) made by any one or more of the methods of Aspects 1 to 9, the substituted poly(alkylene oxide) of Aspect 10 or 11, or the surfactant composition of any one or more of Aspects 12 to 14.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt %, or, more specifically, 5 to 20 wt %”, is inclusive of the endpoints and all intermediate values of the ranges of “5 to 25 wt %,” etc.). “Combinations” is inclusive of blends, mixtures, alloys, reaction products, or the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements can be combined in any suitable manner in the various embodiments.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group.

As used herein, the term “hydrocarbyl” and “hydrocarbon” refers broadly to a substituent comprising carbon and hydrogen, optionally with 1 to 3 heteroatoms, for example, oxygen, nitrogen, halogen, silicon, sulfur, or a combination comprising at least one of the foregoing; “alkyl” refers to a straight or branched chain, saturated monovalent hydrocarbon group; “alkylene” refers to a straight or branched chain, saturated, divalent hydrocarbon group; “alkylidene” refers to a straight or branched chain, saturated divalent hydrocarbon group, with both valences on a single common carbon atom; “alkenyl” refers to a straight or branched chain monovalent hydrocarbon group having at least two carbons joined by a carbon-carbon double bond; “cycloalkyl” refers to a non-aromatic monovalent monocyclic or multicylic hydrocarbon group having at least three carbon atoms, “cycloalkenyl” refers to a non-aromatic cyclic divalent hydrocarbon group having at least three carbon atoms, with at least one degree of unsaturation; “aryl” refers to an aromatic monovalent group containing only carbon in the aromatic ring or rings; “arylene” refers to an aromatic divalent group containing only carbon in the aromatic ring or rings; “alkylaryl” refers to an aryl group that has been substituted with an alkyl group as defined above, with 4-methylphenyl being an exemplary alkylaryl group; “arylalkyl” refers to an alkyl group that has been substituted with an aryl group as defined above, with benzyl being an exemplary arylalkyl group; “acyl” refers to an alkyl group as defined above with the indicated number of carbon atoms attached through a carbonyl carbon bridge (—C(═O)—); “alkoxy” refers to an alkyl group as defined above with the indicated number of carbon atoms attached through an oxygen bridge (—O—); and “aryloxy” refers to an aryl group as defined above with the indicated number of carbon atoms attached through an oxygen bridge (—O—).

Unless otherwise indicated, each of the foregoing groups can be unsubstituted or substituted, provided that the substitution does not significantly adversely affect synthesis, stability, or use of the compound. The term “substituted” as used herein means that at least one hydrogen on the designated atom or group is replaced with another group, provided that the designated atom's normal valence is not exceeded. When the substituent is oxo (i.e., ═O), then two hydrogens on the atom are replaced. Combinations of substituents or variables are permissible provided that the substitutions do not significantly adversely affect synthesis or use of the compound. Exemplary groups that can be present on a “substituted” position include, but are not limited to, cyano; hydroxyl; nitro; azido; alkanoyl (such as a C₂₋₆ alkanoyl group such as acyl); carboxamido; C₁₋₆ or C₁₋₃ alkyl, cycloalkyl, alkenyl, and alkynyl (including groups having at least one unsaturated linkages and from 2 to 8, or 2 to 6 carbon atoms); C₁₋₆ or C₁₋₃ alkoxys; C₆₋₁₀ aryloxy such as phenoxy; C₁₋₆ alkylthio; C₁₋₆ or C₁₋₃ alkylsulfinyl; C₁₋₆ or C₁₋₃ alkylsulfonyl; aminodi(C₁₋₆ or C₁₋₃)alkyl; C₆₋₁₂ aryl having at least one aromatic rings (e.g., phenyl, biphenyl, naphthyl, or the like, each ring either substituted or unsubstituted aromatic); C₇₋₁₉ arylalkyl having 1 to 3 separate or fused rings and from 6 to 18 ring carbon atoms; or arylalkoxy having 1 to 3 separate or fused rings and from 6 to 18 ring carbon atoms, with benzyloxy being an exemplary arylalkoxy.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method for the synthesis of a substituted poly(alkylene oxide), the method comprising:
reacting a substituted alcohol of formula (1) 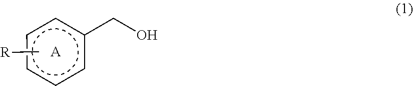
with an alkylene oxide of formula (2) 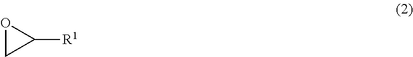
in the presence of a catalyst and under conditions effective to provide the substituted poly(alkylene oxide) of formula (3) 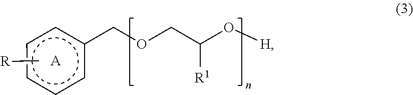
wherein in the foregoing formulas,
each R is independently hydrogen, C₁₋₆₀ alkyl, or C₃₋₁₂ cycloalkyl,
ring A is cyclohexane or phenyl,
each R¹ is independently hydrogen, methyl, ethyl, propyl, butyl, hexyl, decyl, dodecyl, tetradecyl, or hexadecyl, and
n is 2 to 60.

2. The method of claim 1, wherein the catalyst is a base, a double metal cyanide, or a calcium compound.

3. The method of claim 1, wherein
the catalyst is sodium hydroxide, sodium methoxide, sodium ethoxide, magnesium oxide, potassium hydroxide, cesium hydroxide, strontium hydroxide, barium hydroxide, or barium oxide; or
the catalyst is a double metal cyanide compound of formula (4)
M¹ₐ[M²(CN)_{b}L_{c}]_{d} (4)
wherein
M¹ is Zn, Fe, Ni, Co, Mn, Sn, Pb, Mo, Al, V, Sr, W, Cu, or Cr,
M² is Fe, Co, Cr, Mn, V, Ir, Ni, Rh, or Ru,
L is a halogen, NO, NO₂, CO, OH, H₂O, NCO, or NCS,
a is 1 to 3,
b is 5 or 6,
c is 0 or 1, and
d is 1 or 2; or
the catalyst is calcium oxide, calcium hydroxide, calcium sulfate, a calcium alkoxide, calcium acetate, calcium benzoate, calcium butyrate, calcium cinnamate, calcium citrate, calcium formate, calcium isobutyrate, calcium lactate, calcium laurate, calcium linoleate, calcium oleate, calcium palmitate, calcium propionate, calcium stearate, calcium valerate, calcium hexanoate, calcium octanoate, or a combination comprising at least one of the foregoing.

4. The method of claim 1, wherein the catalyst is zinc hexacyanoferrate (III), zinc hexacyanoferrate (II), nickel (II) hexacyanoferrate (II), nickel (II) hexacyanoferrate (III), zinc hexacyanoferrate (III) hydrate, cobalt (II) hexacyanoferrate (II), nickel (II) hexacyanoferrate (III) hydrate, ferrous hexacyanoferrate (III), cobalt(II) hexacyanocobaltate (III), zinc hexacyanocobaltate (III), zinc hexacyanomanganate (II), zinc hexacyanochromate (III), zinc iodopentacyanoferrate (III), cobalt (II) chloropentacyanoferrate (II), cobalt (II) bromopentacyanoferrate (II), iron (II) fluoropentacyanoferrate (III), iron (III) hexacyanocobaltate (III), zinc chlorobromotetracyanoferrate (III), iron (III) hexacyanoferrate (III), aluminum dichlorotetracyanoferrate (III), molybdenum (IV) bromopentacyanoferrate (III), molybdenum (VI) chloropentacyanoferrate (II), vanadium (IV) hexacyanochromate (II), vanadium (V) hexacyanoferrate (III), strontium (II) hexacyanomanganate (III), tungsten (IV) hexacyanovanadate (IV), aluminum chloropentacyanovanadate (V), tungsten (VI) hexacyanoferrate (III), manganese (II) hexacyanoferrate (II), chromium (III) hexacyanoferrate (III), zinc hexacyanoiridate (III), nickel (II) hexacyaniridate (III), cobalt (II) hexacyanoiridate (III), ferrous hexacyanoiridate (III), or a combination comprising at least one of the foregoing.

5. The method of claim 1, wherein the catalyst further comprises an activator that is an alcohol, an aldehyde, a ketone, an ether, an amide, a urea, a nitrile, a sulfide, or a combination comprising at least one of the foregoing.

6. The method of claim 1, wherein each R is independently hydrogen or C₁₋₁₆ alkyl.

7. The method of claim 1, wherein each R is independently hydrogen or C₃₋₈ cycloalkyl.

8. The method of claim 1, wherein the substituted poly(alkylene oxide) is of formula (3a) 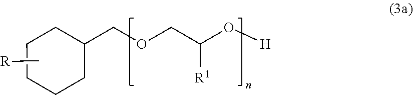
wherein,
each R is independently hydrogen or C₁₋₁₆ alkyl,
each R¹ is independently hydrogen or methyl, and
n is 2 to 32.

9. The method of claim 1, further comprising reacting a phenyl compound of formula (5) 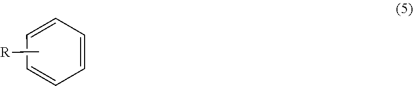
with acetic anhydride or acetyl chloride to provide an acetophenone compound of formula (6) 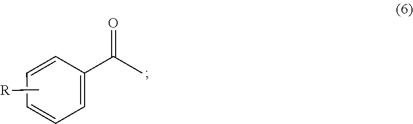
oxidizing the acetophenone compound to provide a substituted benzoic acid compound of formula (7) 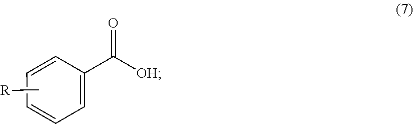
and
reducing the substituted benzoic acid compound to provide a substituted alcohol of formulas (1a) or (1b): 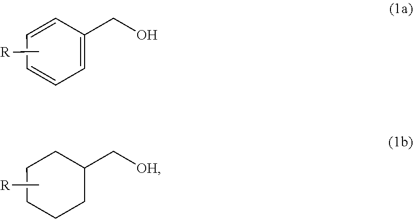
or a combination comprising at least one of the foregoing.

10. A substituted poly(alkylene oxide) made by the methods of claim 1.

11. The substituted poly(alkylene oxide) of claim 10, wherein
the substituted poly(alkylene oxide) has at least one of
a hydrophile-lipophile balance number of 7 to 14;
a critical micelle concentration of 0.01 to 5%, as measured at 25° C.;
a dynamic surface tension of 10 to 75 dynes per centimeter, as measured at 1% actives at 25° C.;
a pour point of −40 to 20° C.; or
a viscosity of 20 to 50 centipoise, as measured at 25° C.

12. A surfactant composition comprising the substituted poly(alkylene oxide) of claim 10.

13. The surfactant composition of claim 12, wherein the surfactant composition comprises the substituted poly(alkylene oxide) in an amount of 0.1 to 50 weight percent, based on a total weight of the surfactant composition.

14. The surfactant composition of claim 12, further comprising an auxiliary surfactant, a solvent, an enzyme, an enzyme stabilizer, a viscosity regulator, a bleach, a hydrotope, an inorganic salt, a fragrance, a dye, a buffer, a preservative, a nutrient, a moisturizer, an emollient, or a combination comprising at least one of the foregoing.

15. A method for formulating and producing a cleaning product or a personal care product, the method comprising using the substituted poly(alkylene oxide) made by the methods of claim 1.

16. The method of claim 5, wherein the catalyst is a base, a double metal cyanide, or a calcium compound; and wherein each R is independently C₁₂ alkyl or C₁₆ alkyl.

17. The method of claim 5, wherein the catalyst is a base, a double metal cyanide, or a calcium compound; and wherein each R is independently cyclopentyl, cyclohexyl, or cycloheptyl.

18. The method of claim 8, wherein the catalyst is a base, a double metal cyanide, or a calcium compound; and wherein each R is independently C₁₂ alkyl or C₁₆ alkyl.

19. The substituted poly(alkylene oxide) of claim 10, wherein the substituted poly(alkylene oxide) has at least one of
a hydrophile-lipophile balance number of 9 to 11;
a critical micelle concentration of ably 0.01 to 1%, as measured at 25° C.;
a dynamic surface tension of 10 to 40 dynes per centimeter, as measured at 1% actives at 25° C.;
a pour point of −20 to 0° C.; or
a viscosity of 25 to 35 centipoise, as measured at 25° C.

20. A method for formulating and producing a cleaning product or a personal care product, the method comprising using the surfactant composition of claim 13.
